(19) United States
(12) Patent Application Publication
(10) Pub. No.: US 2005/0171604 A1
(43) Pub. Date: Aug. 4, 2005

(54) Unicondylar knee implant

(76) Inventor: Alexander Michalow, Bourbonnais, IL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/038,785

(22) Filed: Jan. 19, 2005

Related U.S. Application Data
(60) Provisional application No. 60/537,571, filed on Jan. 20, 2004.

(51) Int. Cl. A61F 2/38; A61F 2/30

(52) U.S. Cl. 623/14.12; 623/20.28; 623/23.41

(57) ABSTRACT

A knee prosthesis, methods of implanting the prosthesis, method of treating arthritis of the knee, and a kit therefore are provided. The prosthesis answers many of the limitations of current knee prosthetic devices by providing a two-component (or alternatively, an optional three-component) device, as either a single structure, or as separate pieces. One of the components is constructed of low friction material, while the second is composed of a weight-dissipating cushioning material; the optional third component is constructed of low friction material. The prosthesis is initially attached to surrounding soft tissue in the knee by biodegradable sutures; it is held permanently in place by fibrous ingrowth into a porous collagen rim in the cushioning component. Major improvements provided by the present invention over currently available prostheses include minimal incisions, minimal or no bone cuts, minimal overall dissection (these improvements lead to shorter hospital stays and rapid rehabilitation and fewer potential for side effects), less prosthetic wear, greater longevity, fewer activity restrictions, able to be used on young, large, active patients, ease of revision, ease of conversion into a total knee arthroplasty if needed.

This application claims the benefit of priority from U.S. provisional application 60/537,571, filed Jan. 20, 2004, which is hereby incorporated by reference in its entirety.

### FIELD OF INVENTION

The present invention relates to the field of prosthetic devise for human joints. The prosthetics are used for partial or total joint replacement, of for the treatment of chronic conditions such as arthritis. The present invention relates to a prosthesis for the human knee, methods of implanting the prosthesis, a kit for facilitating the implantation of the prosthesis, and a method for manufacturing the prosthesis.

### BACKGROUND OF THE INVENTION

The knee joint is divided into three compartments. The medial and lateral compartments are the weight bearing compartments, while the patello-femoral (PF) compartment articulates the patella with the underlying femur, the patella acting as a pulley for the knee extension/quadriceps muscle mechanism. The surfaces of the joint are covered with cartilage, which has two main functions: it both provides a low-friction (LF) bearing surface and acts to absorb and dissipate the loads that are associated with activities such as walking and running.

The knee joint has two types of cartilage, hyaline and meniscal. Hyaline cartilage is attached to the femur, tibia and patella. Meniscal cartilage is a fibrous type of cartilage; in the knee are found a medial and lateral meniscus, two C-shaped structures, one in each of the medial and lateral compartments, which help absorb the loads that occur with weight-bearing activities.

Over time, and with injury or overuse, cartilage breaks down. Unfortunately, cartilage has relatively little capacity for repair. As it breaks down the body's natural healing response is activated; however, instead of healing, chronic inflammation occurs. This inflammation in turn causes pain, which is better known as arthritis. Once arthritis sets in a person is susceptible to chronic pain. When the degeneration of the cartilage progresses beyond a tolerable level of pain the joint can be replaced with a prosthesis in order to relieve the pain. A joint prosthesis replaces the degenerated cartilage with artificial components, generally made out of metals, ceramics, plastics and/or elastomers.

Knee prosthetic devices can be divided into several types, the most common of which is called a total knee arthroplasty (TKA). The TKA replaces all three compartments of the knee. The femur is replaced with one large component that covers the entire medial, lateral and PF compartments. The tibia is covered by one large tibial component. In between the femoral and tibial components, a plastic (often ultra-high molecular weight polyethylene (UHMWPE)) component is inserted and generally secured to the tibial component. The femoral component articulates with the UHMWPE component that is secured to the tibial component. The patellar surface is generally replaced by a UHMWPE patellar “button” component.

There are several technical problems associated with TKAs. Among these is the fact that UHMWPE undergoes wear over time. The microscopic wear particles that are formed incite inflammation and loosening of all the components, which in turn ultimately requires a revision surgery. TKAs must also be inserted properly, including maintaining ligament tension balance and proper mechanical alignment of the components; when these are not performed properly the rate of eventual wear is higher than normal. Additionally, the procedure itself is very stressful to the patient, requiring several months, or longer, of rehabilitation before full strength and function are regained. Generally speaking, at least 3 days are spent in the hospital.

TKAs wear more rapidly in young, active patients. Thus, the procedure is usually delayed in young (i.e. less than 50 year-old) patients. These patients must either wait, enduring the accompanying pain, or, alternatively, they may undergo a TKA, with the likelihood that a second procedure will be required 5 to 20 years later. Finally, once a TKA has been performed, there are certain limits to patient's athletic activities, an additional drawback for the active patient wanting to continue such activities.

Not all patients have arthritic degeneration in all three knee compartments. Many, especially young, patients, generally have degeneration in only one or two compartments. Due to this fact, a uni-compartmental knee arthroplasty (UKA) is sometimes performed. In the most common type of UKA, the medial compartment is replaced with a prosthesis, sparing the lateral and PF compartments from surgical dissection. The advantage to such a procedure is that there is much less surgery involved, leading to a shorter hospital stay and much more rapid rehabilitation. However, this type of prosthesis has the same problems as does a TKA, in that UHMWPE wear and loosening occurs. In addition, the tibial component may subside, leading to failure of the prosthesis. Again, athletic activities must often be curtailed, in order to prevent subsidence of the tibial component and increased wear of the UHMWPE. This limitation of activity is necessary to prolong the useful life of the prosthesis.

Although lateral UKAs and PF replacements are currently available, they do not have the same generally good, reproducible results of the medial UKA. Additionally, lateral UKAs and PF replacements have the same drawbacks as do TKAs and medial compartment UKAs.

Another type of replacement in the knee is a meniscal replacement, a device meant to replace a torn or degenerating meniscus. These devices may be completely synthetic, synthetic with fibrous ingrowth at the periphery, or a scaffold for cellular ingrowth with an eventual meniscus made out of collagen and autologous cells.

Meniscal replacements that are made out of synthetic material and not meant for cellular ingrowth are represented by U.S. Pat. Nos. 4,502,161 (the '161 patent); U.S. Pat. No. 5,171,322 (the '322 patent); and U.S. Pat. No. 5,344,459 (the '459 patent). The '161 patent describes a meniscal replacement made out of a woven fiber with an outer resilient coating; the device is anchored by a screw at the side of the tibia. The '322 patent describes a stabilized meniscus replacement. The patent does not state specific material; it merely indicates that the prosthesis may be made out of a “biocompatible resilient material.” The '459 patent describes an arthroscopically implantable meniscus replacement, a donut-shaped polymeric device meant to cushion the articulation in an arthritic joint, preferably the knee joint. The implant is made from any one of several materials, including polyethylene, polypropylene, polyurethane or polybutyl rubber.

Meniscal replacements made out of synthetic material, with a porous periphery allowing for fibrous ingrowth to facilitate attachment to surrounding soft tissue are represented by U.S. Pat. Nos. 4,919,667 (the '667 patent); U.S. Pat. No. 4,344,193 (the '193 patent); and U.S. Pat. No. 6,629,997 (the '997 patent). These patents are hereby incorporated by reference in their entirety. The '667 patent describes a meniscus implant made out of woven fiber and a bonding material, with a porous coating allowing for fibrous ingrowth to anchor the prosthesis to surrounding tissue. The '193 patent describes a meniscus which is made out of silicone rubber, potentially with a porous border to allow for fibrous ingrowth. The '997 patent describes a meniscal implant with a hydrogel surface, reinforced by a 3D mesh. The mesh of this implant is interwoven in a hydrogel for strength, where the hydrogel articulates against adjacent joint surfaces; surrounding tissue may or may not ingrow into the implant at its periphery. This particular implant does not use a low-friction material meant to articulate against adjacent joint surfaces, but rather uses a soft hydrogel. Additionally, the patent claims the use of a mixture of a soft hydrogel and a relatively harder hydrogel; the soft component is intended for joint articulation and the harder hydrogel is meant for the interior portion of the device. The patent does not disclose an implant made for an arthritic joint, but rather one meant for replacement of damaged meniscal tissue.

A third type of meniscus replacement is the kind made out of material that allows for cellular and fibrous ingrowth, eventually forming a new meniscus made out of normal collagen tissue that was synthesized by the autologous cells that “invaded” the scaffold. U.S. Pat. Nos. 4,880,429, 5,007,934, and 5,158,574 are representative of this type of device.

A major limitation of all of these meniscal replacement devices is that they do not replace hyaline cartilage. In an arthritic degenerating joint both meniscal and hyaline cartilage are damaged. The above-mentioned meniscal replacements do not replace the damaged hyaline cartilage, only meniscal cartilage, and thus these devices are not suitable for an arthritic joint replacement. Furthermore, these devices do not have any low-friction bearing surfaces which mimic the low-friction bearing function of hyaline cartilage; they merely act as cushioning devices.

Another type of knee implant is known as a knee spacer. This type of implant is meant to replace more than the meniscal cartilage; it is generally indicated for replacement of a degenerating joint. U.S. Pat. No. 4,052,753 describes a surgically implantable knee prosthesis; the device is essentially a supra-patellar knee spacer. Most knee spacers, however, relate to the tibio-femoral articulation. In fact, several of the meniscal replacements referenced above are actually knee spacer devices that are called meniscal replacements.

U.S. Pat. No. 6,206,927 describes a surgically implantable knee prosthesis which is a tibio-femoral knee spacer device. It is marketed and distributed as the UniSpacer™ device by Sulzer, Inc. The UniSpacer™ device was developed in order to avoid the wear problems associated with polyethylene devices in young active patients with single compartment degeneration. The design of the UniSpacer™ device is based on three premises: correction of the mechanical deformity and replacement of the missing articular material with the implant; replacement of the meniscal function by a translational and rotational load bearing material; and maintenance of correct anatomical kinematics and restored ligament tension throughout the range of motion. The prosthesis consists of a metal, ceramic, or polymer material. It is meant to occupy the space between the tibial plateau and the respective femoral condyle.

The implantation of tibio-femoral spacers was originally devised by McKeever in 1957 (Figueroa, Luis, et al., from the course on Mechanics of Materials-I, Applications of Engineering Mechanics in Medicine, GED-University of Puerto Rico, Mayaguez, Engineering Biomechanics of Bone and Artery Replacement, May 2004, p. 2.) and later by Macintosh in 1958 (Macintosh, Hemiarthroplasty of the knee using a space occupying prosthesis for painful varus and valgus deformities. Proceedings of the Joint Meeting of Orthopaedic Associations of the English-Speaking World, JBJS 40(A), December 1958:1431). The devices were developed because of problems associated with the original knee prosthetic devices that were attached to bone, developed in the 30s and 40s. These original devices were hinged, and, although they provided relatively good short-term results, they demonstrated poor range of motion and showed severe problems with loosening and infection. For these reasons they were abandoned and the McKeever and Macintosh devices were adopted. These devices demonstrated some success in pain relief, but results were not predictable. Total knee replacements were developed because many patients continued to show symptoms. In 1968 the first metal and plastic knee, secured to bone with cement, was developed. Later, in 1972, Insall designed what has become the prototype for current TKAs.

The problems associated with current TKAs primarily involve wear and/or loosening of the prosthetic components, which are often especially pronounced in, and of concern to, young and active patients. When revisions are needed, a major problem is the loss of bone, poorer results than obtained in the original surgery, etc.; these problems can occur regardless of patient age.

Many patients (especially younger ones) with arthritis may have only a single compartment (more often medial vs. lateral) involved with the arthritic degeneration. If such a patient required replacement surgery it would be advantageous to have a procedure in which only the degenerated compartment is replaced. Thus, in order to treat single compartment degenerative disease, uni-compartmental knee arthroplasty (UKA) was developed. Currently, UKA is optimized for the medial compartment. In older designs a major disadvantage of UKA prostheses was that a follow-up TKA was often more difficult to perform, and the TKA results were often compromised. More recent UKAs are designed with the concept of preserving tibial bone so as not to lead to a comprised TKA in the future.

There are several advantages to such a device. It is relatively easy to insert and is also easy to remove, especially if degeneration develops in other compartments in the future, or if infection sets in. The UniSpacerm device is based on the fact that no bone resection is needed for its insertion, thus bone cuts are not required for proper implant function, though shaving of the tibial surface may indicated. Instead, the implant adapts to the kinematics of the knee. Furthermore, because no bone is resected future TKAs are not complicated. By avoiding cutting the medial tibial bone, the load bearing capacity of the medial compartment is not compromised. Loosening is not likely as a possible mode of failure because the device is not attached to bone.

In spite of the advantages of such an implant, the UniSpacer™ device has several problems associated with it. Of major concern is the fact that it does not relieve all a patient's pain. The product is marketed as a device that relieves only some of the pain, in anticipation of a TKA in the future. It is only indicated for the relatively younger patient with unicompartmental disease who wants to maintain a high level of activity, but is willing to live with some pain, even after this device is inserted.

The ABS, Inc. InterCushion™ device is a second type of unattached spacer device, and is meant to be placed between arthritic femoral and tibial surfaces. It resembles the UniSpacer™ device in that it is shaped to fit between the two joint surfaces. This device, however, is not made out of a rigid material such as metal. Instead, it is made out of an elastomer, polyurethane. The advantage of this device is that it acts as a cushion, and dissipates stresses between the joint surfaces. With better stress dissipation it is expected that there would be less post-operative pain than that associated with the UniSpacer™ device. The InterCushion™ device is not, however, a low-friction implant.

Bonutti describes yet another type of device that is similar to the above knee spacers in U.S. Pat. No. 6,770,078. In this device the final implant is unattached to surrounding tissues. It is designed such that it is free to move about the tibial surface, allowing for 360° of rotation. However, this implant requires two surgical procedures. In the first procedure a biodegradable implant is sutured to surrounding ligaments, allowing for tissue ingrowth. After a period of time, a ‘wall’ of tissue forms at the periphery of the biodegraded implant, which then acts to contain the final implant, which is inserted at the time of the second surgical procedure. It is a disadvantage for the patient that this implant requires two surgical procedures. Additionally, while this invention describes the use of low-friction material such metal, ceramic, and/or porous materials, it does not include the use of any elastomeric materials.

Accordingly, while conventional implants are useful, they have numerous significant disadvantages in their use; thus a need remains for a prosthesis that uses a combination of materials to achieve both a low-friction surface and a cushioning function to dissipate force.

### SUMMARY OF THE INVENTION

A knee prosthesis, methods of implanting the prosthesis, method of treating arthritis of the knee, and a kit therefore are provided. The prosthesis answers many of the limitations of current knee prosthetic devices by providing a two-component (or optionally, a three component) device, as either a single structure, or as separate pieces. One of the components is constructed of low friction material, while the second is composed of a weight-dissipating cushioning material; the optional third component is constructed of low friction material. The prosthesis is initially attached to surrounding soft tissue in the knee by biodegradable sutures; it is held permanently in place by fibrous ingrowth into a porous collagen rim in the cushioning component. Major improvements provided by the present invention over currently available prostheses include minimal incisions, minimal or no bone cuts, minimal overall dissection (these improvements lead to shorter hospital stays and rapid rehabilitation and fewer potential for side effects), less prosthetic wear, greater longevity, fewer activity restrictions, able to be used on young, large, active patients, ease of revision, ease of conversion into a total knee arthroplasty if needed.

Knee arthritis is treated with an implant that mimics the function of both meniscus and hyaline cartilage in a knee joint. The implant replaces the two major functions of these two cartilage types, including low friction articulation and weight load dissipation (cushioning). This is accomplished by the use of two materials. The low-friction aspect is accomplished by the use of a low-friction, hard material. The cushioning property is accomplished by the use of an elastomeric compound. The implants are designed such that surgical dissection is minimized. There is either no or minimal bone resection. No component is attached to the tibial surface. The cushioning component essentially glides on the tibial surface, being attached at its periphery by, initially, biodegradable sutures, and permanently, by fibrous ingrowth from the surrounding soft tissues, as the normal meniscus. The implants include separate medial and/or lateral uni-compartmental implants. The femoral portion of the implant may either be unattached to the femoral condyle, or it may be attached to the condyle. In the former case, the unattached low friction unit is actually attached to the cushioning component, and the combined two-material unit glides on the tibia. In this case the femoral condyle articulates against the underlying low friction portion of the implant. In the latter case, because the low friction component is attached to the femoral condyle, it articulates against the cushioning portion of the implant. The cushioning component is unattached and essentially acts as a cushion between the two joint surfaces. In order to decrease friction between this implant and the underlying tibial surface, an additional option is to have a thin layer of the low friction material attached to the undersurface, or lower surface, of the cushioning component, such that there would be a low amount of friction between the mobile cushioning implant and the underlying tibial articular surface. A final option is to use hyaluronic acid-coated surfaces on the implants in order to further decrease friction and provide a more biological bearing surface.

The implant of the present invention mimics the function of both meniscus and hyaline cartilage in a knee joint. It replaces the two major functions of these two cartilage types, including low friction articulation and weight load dissipation (cushioning). This is accomplished by the use of two materials. The low-friction aspect is accomplished by the use of a low-friction, hard material. The cushioning property is accomplished by the use of an elastomeric compound. The implants are designed such that surgical dissection is minimized. There is either no or minimal bone resection. No component is attached to the tibial surface. The cushioning component essentially glides on the tibial surface, being attached at its periphery by, initially, biodegradable sutures, and permanently, by fibrous ingrowth from the surrounding soft tissues, similar to the attachment of the normal meniscus to the surrounding menisco-tibial ligaments. The implant may have capacity for fibrous ingrowth from surrounding soft tissue all around the periphery, or on only a portion of the periphery, including the anterior, medial/lateral, and/or posterior portions of the implant. The implants include separate medial and/or lateral uni-compartmental implants. The femoral portion of the implant may either be unattached to the femoral condyle, or it may be attached to the condyle. In the former case, the unattached low friction unit is actually attached to the cushioning component, and the combined two-material unit glides on the tibia. In this case the femoral condyle articulates against the underlying low friction portion of the implant. In the latter case, because the low friction component is attached to the femoral condyle, it articulates against the cushioning portion of the implant. The cushioning component is unattached to tibial bone, and is attached only to surrounding soft tissues at its periphery, and essentially acts as a cushion between the two joint surfaces. In order to decrease friction between this implant and the underlying tibial surface, an additional option is to have a thin layer of the low friction material attached to the undersurface of the cushioning component, such that there would be a low amount of friction between the mobile cushioning implant and the underlying tibial articular surface. A final option is to use hyaluronic acid-coated surfaces on the implants in order to further decrease friction and provide a more biological bearing surface. This invention overcomes many of the problems associated with knee prosthetic devices in the past, which include extensive incisions, extensive bone cuts, extensive overall dissection, long hospital stays, slow rehabilitation, high potential for side effects, great prosthetic wear, poor longevity, prosthetic loosening, extensive activity restrictions, poor performance in young, large, active patients, difficulty of revision, and difficulty of conversion into a total knee arthroplasty if needed.

In accordance with the present invention, there are a number of embodiments herein disclosed.

Thus in one embodiment of the present invention, a prosthetic device is provided as a single structure, comprising two components: an upper low friction layer and a lower cushioning layer. It is intended that the prosthetic device not be attached to the tibia or the femur. The upper layer is made out of a low friction material. Bound to the undersurface, or lower surface, of the upper layer is the elastomeric cushioning component (CC). The upper, low friction layer is called the femoral low friction component (FLFC). It is contoured to match the shape of the femoral condyle. The CC, which is made out of an elastomeric material, is contoured on its superior, or upper, surface to the exact dimensions of the undersurface, or lower surface, of the FLFC in order that the two could be attached. The undersurface, or lower surface,of the CC is generally flat with a slight convexity, in order to coincide with the relatively flat, slightly convex tibial articular surface. The contour is given a slight variation in order to better mimic the shape of the medial vs. the lateral tibial surface geometry.

In an aspect of this embodiment, the FLFC is made from a material selected from the group comprising metal, metal alloy with an amorphous atomic structure (of which Liquidmetal® alloys from Liquidmetal® Technologies of Lake Forest, Calif. are representative), ceramic, glass, carbon composites, polymers, ceramic-coated surface materials, diamond-coated surface materials, or pyrolitic carbon-coated surface materials.

In yet another aspect, the FLFC is made from metal. In a preferred aspect the metal is selected from the group comprising stainless steel, titanium, or cobalt-chrome alloy.

In yet another aspect, the FLFC is made from ceramic. In a preferred aspect the ceramic is selected from the group comprising alumina, or zirconium oxide.

In yet another aspect, the FLFC is made from carbon composite. In a preferred aspect the carbon composite is P25-CVD.

In yet another aspect, the FLFC is made from a polymer. In a preferred aspect the polymer is selected from the group comprising polyetheretherketone, polyetherketoneketone, polyaryletherketone, or polysulfone.

In yet another aspect, the FLFC is made from a polymer optionally reinforced with fiber.

In yet another aspect, the FLFC is made from pyrolitic-carbon coated material.

In yet another aspect, the FLFC is made from a ceramic-coated material.

In yet another aspect, the FLFC is made from a diamond-coated material.

In yet another aspect, the FLFC is made from glass.

In yet another aspect, the FLFC is made from metal alloy with an amorphous atomic structure (of which Liquidmetal® alloys from Liquidmetal® Technologies of Lake Forest, Calif. are representative). In a preferred aspect, the alloy is selected from the group comprising titanium-based Liquidmetal® alloy or zirconium-based Liquidmetal® alloy. In an even more preferred aspect the alloy is zirconium-based Liquidmetal® alloy.

In yet another aspect, the CC is made from an elastomeric material selected from the group comprising polyurethane, polyvinylalcohol, polyacrlyamide, or fiber-reinforced polymer. In a preferred aspect the CC is made from polyurethane.

In yet another aspect, the CC is made from a capsule comprising a water retaining center surrounded by a supportive outer covering. In a preferred aspect, the water retaining center is made from hydrogel material selected from the group comprising polyacrylamide or polyvinylalcohol.

In yet another aspect, the prosthesis is suitable for attachment to surrounding soft tissue by the entire periphery of the implant. In a preferred aspect, the prosthesis is attached to the menisco-tibial ligaments.

In yet another aspect, the prosthesis is suitable for attachment to surrounding soft tissue by only a portion of the periphery of the implant, including the anterior, medial/lateral, and/or posterior portion(s) of the implant. In a preferred aspect, the prosthesis is attached to the menisco-tibial ligaments.

In yet another aspect, the prosthesis is suitable for initial attachment to surrounding soft tissue by glue or sutures.

In yet another aspect, the CC further comprises a porous collagen ingrowth coating that facilitates permanent attachment via fibrous ingrowth.

In yet another aspect, the FLFC is contoured to approximate the shape of the femoral condyle.

In yet another aspect, the FLFC has a radius of curvature equal to or larger than that of the femoral condyle against which it is intended to articulate. In a preferred aspect, the FLFC has a radius of curvature greater than that of the femoral condyle against which it is intended to articulate.

In yet another aspect, the superior surface of the CC is contoured to exactly match the undersurface of the FLFC.

In yet another aspect, the CC is slightly larger than the FLFC.

In yet another aspect, the CC is attached to the FLFC by mechanical interdigitation, glue, or other bonding method.

In yet another aspect, the CC is attached to the FLFC prior to packaging.

In yet another aspect, the CC is attached to the FLFC immediately prior to implantation. In a preferred aspect, the method of attachment of the CC to the FLFC is by mechanical interlocking fixation. In a more preferred aspect, the method of attachment is by a snapping mechanism.

In yet another aspect, the prosthesis comprising a single structure, of three components: an upper low friction layer, a middle cushioning layer and a lower low-friction layer; wherein it is intended that the prosthetic not be attached to the tibia or the femur; the upper layer is made out of a low friction material; bound to the undersurface of the upper layer is the elastomeric cushioning component (CC); the upper, low friction layer is called the femoral low friction component (FLFC); it is contoured to match the shape of the femoral condyle; the CC, which is made out of an elastomeric material, is contoured on its superior surface to the exact dimensions of the undersurface of the FLFC in order that the two could be attached; the undersurface of the CC is generally flat with a slight convexity, in order to coincide with the relatively flat, slightly convex tibial articular surface; the contour is given a slight variation in order to better mimic the shape of the medial vs. the lateral tibial surface geometry; further comprises a tibial low friction component (TLFC), said superior, or upper, surface of component being attached to the undersurface of the cushioning component.

In yet another aspect, the TLFC is attached to the cushioning component-femoral low friction component unit by mechanical interdigitation, glue, or other bonding method.

In yet another aspect, the TLFC is attached to the cushioning component-femoral low friction component unit prior to packaging.

In yet another aspect, the TLFC is attached to the cushioning component-femoral low friction component unit immediately prior to implantation. In a preferred aspect, the method of attachment of the TLFC to the CC is by mechanical interlocking fixation. In a more preferred aspect, the method of attachment is by a snapping mechanism.

In yet another aspect, the prosthesis components are optionally coated with hyaluronic acid.

In yet another aspect, the FLFC is suitable for attachment to the femoral condyle. In a preferred aspect, the FLFC is suitable for attachment to the femoral condyle by bone cement, or by use of a porous coating, and/or a hydroxy-apatite coating on the implant which allows for bone ingrowth into the implant.

In yet another aspect, the FLFC is coated with an elastomeric or cushioning material (e.g. polyurethane).

In another embodiment of the present invention, a prosthetic device is provided as two components which are not attached to each other: an upper low friction layer and a lower cushioning layer. It is intended in this embodiment that the prosthesis not be attached to the tibia, but one component is attached to the femur. The upper layer is made out of a low friction material; its superior, or upper, surface is made to attach to the femoral condyle. The upper, low friction layer is called the femoral low friction component (FLFC). Below the upper layer is the elastomeric cushioning component (CC). Its upper surface is contoured to match the shape of the overlying FLFC, against which it articulates. The undersurface of the CC is generally flat with a slight convexity, in order to coincide with the relatively flat, slightly convex tibial articular surface. The contour is given a slight variation in order to better mimic the shape of the medial vs. the lateral tibial surface geometry.

In an aspect of this embodiment, the FLFC is made from a material selected from the group comprising metal, metal alloy with an amorphous atomic structure (of which Liquidmetal® alloys from Liquidmetal® Technologies of Lake Forest, Calif. are representative), ceramic, glass, carbon composites, polymers, ceramic-coated surface materials, diamond-coated surface materials, or pyrolitic carbon-coated surface materials.

In yet another aspect, the FLFC is made from metal. In a preferred aspect the metal is selected from the group comprising stainless steel, titanium, or cobalt-chrome alloy.

In yet another aspect, the FLFC is made from ceramic. In a preferred aspect the ceramic is selected from the group comprising alumina, or zirconium oxide.

In yet another aspect, the FLFC is made from carbon composite. In a preferred aspect the carbon composite is P25-CVD.

In yet another aspect, the FLFC is made from a polymer. In a preferred aspect the polymer is selected from the group comprising polyetheretherketone, polyetherketoneketone, polyaryletherketone, or polysulfone.

In yet another aspect, the FLFC is made from a polymer optionally reinforced with fiber.

In yet another aspect, the FLFC is made from pyrolitic-carbon coated material.

In yet another aspect, the FLFC is made from a ceramic-coated material.

In yet another aspect, the FLFC is made from a diamond-coated material.

In yet another aspect, the FLFC is made from glass.

In yet another aspect, the FLFC is made from metal alloy with an amorphous atomic structure (of which Liquidmetal® alloys from Liquidmetal® Technologies of Lake Forest, Calif. are representative). In a preferred aspect, the alloy is selected from the group comprising titanium-based Liquidmetal® alloy or zirconium-based Liquidmetal® alloy. In an even more preferred aspect the alloy is zirconium-based Liquidmetal® alloy.

In yet another aspect, the CC is made from an elastomeric material selected from the group comprising polyurethane, polyvinylalcohol, polyacrlyamide, or fiber-reinforced polymer. In a preferred aspect the CC is made from polyurethane.

In yet another aspect, the CC is made from a capsule comprising a water retaining center surrounded by a supportive outer covering. In a preferred aspect, the water retaining center is made from hydrogel material selected from the group comprising polyacrylamide and polyvinylalcohol.

In yet another aspect, the prosthesis is suitable for attachment to surrounding soft tissue by the entire periphery of the implant. In a preferred aspect, the prosthesis is attached to the menisco-tibial ligaments.

In yet another aspect, the prosthesis is suitable for attachment to surrounding soft tissue by only a portion of the periphery of the implant, including the anterior, medial/lateral, and/or posterior portion(s) of the implant. In a preferred aspect, the prosthesis is attached to the menisco-tibial ligaments.

In yet another aspect, the prosthesis is suitable for initial attachment to surrounding soft tissue by glue or sutures.

In yet another aspect, the CC further comprises a porous collagen ingrowth coating that facilitates permanent attachment via fibrous ingrowth.

In yet another aspect, the femoral condyle is cut to exactly match the superior surface of the FLFC, which is suitable for binding with bone cement.

In yet another aspect, the femoral condyle is cut to exactly match the superior surface of the FLFC, which is porous coated or hydroxy-apatite coated to allow for bone ingrowth.

In yet another aspect, the undersurface of the FLFC is polished in order to generate a low friction surface.

In yet another aspect, the CC is contoured to exactly match the undersurface of the FLFC.

In yet another aspect, the CC is slightly larger than the FLFC.

In yet another aspect, the prosthesis comprising two components, which are not attached to each other: an upper low friction component, and a single lower component consisting of two materials, a superior cushioning layer attached to a lower low-friction layer; wherein it is intended that the prosthetic not be attached to the tibia, but one component is attached to the femur; the upper low friction component is made out of a low friction material and its superior surface is made to attach to the femoral condyle. The upper, low friction component is called the femoral low friction component (FLFC). Below the upper FLFC layer is the superior part of the lower component, consisting of an elastomeric cushioning component (CC). Its upper surface is contoured to match the shape of the overlying FLFC, against which it articulates. The undersurface of the CC is generally flat with a slight convexity, in order to coincide with the relatively flat, slightly convex tibial articular surface. The contour is given a slight variation in order to better mimic the shape of the medial vs. the lateral tibial surface geometry; further comprises a tibial low friction component (TLFC), said superior surface of said component being attached to the undersurface of the cushioning component.

In yet another aspect, the TLFC is attached to the cushioning component by mechanical interdigitation, glue, or other bonding method.

In yet another aspect, the TLFC is attached to the cushioning component prior to packaging.

In yet another aspect, the TLFC is attached to the cushioning component immediately prior to implantation. In a preferred aspect, the method of attachment of the TLFC to the CC is by mechanical interlocking fixation. In a more preferred aspect, the method of attachment is by a snapping mechanism.

In another aspect, the prosthesis components are optionally coated with hyaluronic acid.

In yet another aspect, the FLFC is suitable for attachment to the femoral condyle. In a preferred aspect, the FLFC is suitable for attachment to the femoral condyle by bone cement or by use of a porous coating, and/or hydroxy-apatite coating on the implant which allows for bone ingrowth into the implant.

In yet another aspect, the FLFC is coated with an elastomeric or cushioning material (e.g. polyurethane).

In another embodiment, there is provided a method of providing a knee prosthesis to a patient in need thereof, said method comprising: ascertaining the size and shape of the required prosthesis and components thereof by examination of the patient; and providing to the patient a prosthesis according to the present invention.

In another embodiment, there is provided a method of knee reconstruction of a patient in need thereof, said method comprising: determining the proper size and shape of a prosthesis and components thereof according to the present invention, by examination of the patient; selecting the prosthesis according to the present invention of said proper size and shape; exposing the knee compartment; and implanting the knee prosthesis into the compartment.

In another embodiment, there is provided a method of making a prosthesis of the present invention comprising CAD/CAM design of molds for casting the prosthesis component.

In yet another embodiment there is provided a method of making a prosthesis of the present invention comprising CAD/CAM techniques to directly machine the components from blocks of material.

In another embodiment, there is provided a kit for treating arthritis of the knee comprising a prosthesis of the present invention and means for implanting said prosthesis.

In another embodiment, there is provided a method of implanting a prosthesis of the present invention, wherein the prosthesis is inserted between the femoral and tibial surfaces.

In another embodiment, numerous sizes of the components are provided so as to provide a prosthetic device appropriate for a given patient.

These and other embodiments of the invention will become apparent in light of the Detailed Description below.

### BRIEF DESCRIPTION OF DRAWINGS

FIG. 1 shows a perspective view of the two piece construct. There is a top, or superior, piece (1), the FLFC (femoral low-friction component), that is made out of a low friction material. Its shape conforms to that of the femoral condyle. This shape resembles the general shape of the meniscus cartilage, but instead of forming a “C” shape with an open central/inner portion as in the normal meniscus, the central or inner portion is solid. The front (anterior) (2), back (posterior) (3), and side (lateral) (4), portions are raised. The undersurface is attached to the elastomeric cushioning component (5).

FIG. 2 shows the manner by which the periphery of the CC is to be attached to the menisco-tibial ligaments, with an area for initial biodegradable suture attachment and permanent fibrous ingrowth. The rim (7) of the CC (5) has a collagen ingrowth coating (7). Rings (8), or a suitable alternative, may be used for suture fixation, which gives initial stability before fibrous ingrowth takes place.

FIG. 3 demonstrates a frontal view of the manner by which the implant is inserted between the femoral and tibial articular surfaces. Fibrous ingrowth from the peripheral menisco-tibial ligaments (10) is demonstrated (9).

FIG. 4 is a lateral view of the manner by which the implant is inserted between the femoral and tibial articular surfaces.

FIG. 5 shows a perspective view of the single unit as a three piece combined construct. Here there is a top, superior, piece (1), the FLFC. The CC has an outer rim for initial biodegradable suture attachment (7) and for later permanent fibrous ingrowth (7).

FIG. 6 demonstrates a lateral view of the attachment of the FLFC (12) to the femoral condyle. It is attached by either the use of bone cement or by bone ingrowth into a porous coated attachment surface on the FLFC (12). Pegs (13) may be added in order to increase fixation stability of the implant into the femoral bone.

FIG. 7 shows the FLFC attached to bone, with the interdigitating CC attached to a TLFC (11) piece at its undersurface. The CC portion may be attached to surrounding soft tissue menisco-tibial ligaments (9) initially by biodegradable sutures and eventually by permanent fibrous ingrowth (10).

FIG. 8A shows the hydrogel/supportive outer coating option for the prosthesis. This cushioning hydrogel is relatively elastic, with a modulus of elasticity (MOE) that is between 0.1-50 MPa. The outer covering is made out of a relatively inelastic material, in order to prevent excessive deformation and to maintain a constant negative inside pressure, such that osmotic flow is always directed inwards. It is preferably made out of material with a relatively low MOE such as ultra high molecular weight polyethylene fibers (MOE @ 700 MPa). There is enough elasticity for bending to occur, but very little stretching occurs. The superior surface has a FLFC as disclosed above. The undersurface has a TLFC, as disclosed above. The CC, instead of being composed of one elastomeric material, may consist of two parts: an inner hydrogel component and an outer water-permeable synthetic fiber component (14). The hydrogel has an affinity for water and will attract water inside, as noted by (15). This constant inward flow of water puts outward pressure on the outer coating (14) and both the FLFC (1) and the TLFC (11), as depicted by the arrows inside the component. This constant inward flow of water is resisted by the outer coating (14).

FIG. 8B shows what would happen if the hydrogel (16) were not surrounded by the outer coating. Here the unimpeded inward flow of water causes the hydrogel to expand to a much larger size. The inward and outward water flow pressures equilibrate (17).

FIG. 8C shows what occurs with weight loads. The weight load (18) causes the thickness of the cushioning component to decrease (19). The outward flow of water increases beyond the inward flow (20).

FIG. 9 shows the hyaluronic acid coating on the prosthesis.

### DETAILED DESCRIPTION OF THE INVENTION

The invention herein relates to a knee prosthetic implant that overcomes some of the limitations of current TKAs, UKAs, and “spacer” devices, methods of implanting the device, and a kit for implantation of the device. The advantages of the device of the current invention include, by way of illustration only but by no means meant to be a comprehensive list, minimizing surgical procedures, minimizing bone dissection, replacement of meniscal cartilage, mimicry of the function of meniscal cartilage, replacement of hyaline cartilage, mimicry of the function of hyaline cartilage, and usefulness for young, active patients with arthritis of the knees for whom TKAs are relatively contraindicated. It is believed that no other current device is available which accomplishes all of mimicry of both meniscal and hyaline cartilage and function, minimal surgical procedure and minimal or no bone cutting, and the potential for attachment to surrounding soft tissue.

The device of the current invention mimics both hyaline and meniscal cartilage function. The knee prosthetic device consists of separate medial and lateral implants. Each implant is designed specifically in a manner that mimics the two main functions of joint cartilage. These two properties are:
(a) Low friction articulation; and
(b) Dissipation of the stresses of weight bearing.

The human body satisfies the above two requirements by the unique interaction of the surface of the cartilage extra-cellular matrix (ECM), with hyaluronic acid acting as a lubricant for low friction articulation, with the flow of water molecules acting to disperse weight bearing stresses. The normal architecture of ECM includes negatively charged proteoglycans (PGs) and a collagen network, both of which have an affinity for water. When a load is applied to cartilage, water is pushed out of the ECM and the negatively charged PGs repel each other, dispersing the load, thus decreasing the load to any one area and to the underlying structures. When the load is released, water flows back into the ECM. This flow of water and the repelling nature of the negatively charged groups are thus responsible for the shock-absorbing properties of cartilage. It is current understanding that the PGs contribute to the compressive and/or swelling properties, while the collagen network provides the cohesive properties (resisting the negatively charged swelling pressure of the PGs) and strength in tension. The importance of this cushioning effect is to dissipate weight-bearing stresses to the joint structures, i.e. cartilage and underlying bone. Without a cushioning effect, there is an increased amount of weight bearing stress that is passed on to local areas of bone; this increased stress to bone may be one of the factors that can lead to pain.

With respect to joint replacement materials, it is difficult, if not impossible, to find a single material, for use in the human body, which provides both low-friction and cushioning. This is because these two properties are in opposition when it comes to mechanical function; the types of materials used to grant either property exemplify this. The best low friction articulating surfaces are generally very hard with little elasticity. Of course, a cushioning effect cannot be provided by a rigid metal device, such as the UniSpacer™ device. Another material which is generally low-friction, ceramic tends to be brittle and thus undergo fatigue failure, which gives it limitations when it is to be used in certain types of implants, and certainly makes it unsuitable for use as a cushioning material. In general, the best bearing surfaces, whether they are ceramic or metal, generally have very low elasticity. Thus the materials with the best bearing surface properties have virtually no, or minimal, stress dissipation (cushioning) effects.

Materials that dissipate stress well inherently have a certain amount of elasticity in them. When stress is applied to the surface of these materials, some motion occurs at the surface; in other words, there is some microscopic movement of the surface molecules. The overall result of this surface action is that it is associated with a higher level of friction when it glides against an opposing surface. Furthermore, this microscopic movement is associated with the development of microscopic particles that break off when an opposing stress is applied to them, i.e. weight bearing stress. Thus, the materials with the best cushioning properties generally do not work well as low friction bearing surfaces.

Although a number of implants have been designed for use as knee replacements for arthritis, there is no single device currently available which exhibits both a low friction surface for articulation and a cushioning component for force dissipation. Current TKAs are designed with a polyethylene implant that is attached to bone, the tibial component, and articulates against a femoral component that is made out of a metal or ceramic. Polyethylene has no elastic or cushioning properties, and thus it does not confer either elasticity or cushioning. U.S. Pat. No. 6,302,916 describes the use of polyurethane in place of polyethylene in a TKA, which is an improvement. However, the TKA procedure requires relatively extensive surgical dissection and bone cuts, and it includes implant attachment to the tibial bone; such extensive surgical requirements do not address the need for minimal surgery. The proposed device of the present invention addresses the needs for a low friction surface, weight dissipating cushioning, and can be inserted with minimal surgery and minimal or no bone cuts, and no attachment to the tibial bone.

One of the problems in standard UKAs is the tibial bone cut. The cut must be made with proper rotation and angulation. Even slightly inaccurate positioning can result in a more rapid rate of wear and loosening. Tibial bone cuts, if made too deep, are associated with subsidence and/or loosening of the tibial component, which leads ultimately to prosthetic failure. Furthermore, by removing some tibial bone, and adding cement into the tibial cancellous bone, a revision TKA becomes more difficult, if one is require in the future.

(a) Low Friction Material

In practicing the invention, the phrase “low friction” means a low coefficient of friction (COF); a low COF in the context of the present invention would be about 0.001 to 0.5; preferably 0.1-0.2 or less. The COF is a ratio of the frictional force resisting movement of an object tangentially to a surface and the force pushing the object into the surface (or normal force). Mathematically, it can be expressed by the formula:
μ=F_{f}÷Fₙ
wherein μ is the COF, F_{f} is the frictional force resisting movement of an object tangentially to a surface, and Fₙ is the normal force.

By way of example, the COF for cartilage on cartilage is 0.001, metal on normal cartilage is 0.05 (but note the COF escalates for metal on degenerative cartilage to 0.25 (Covert, 2001)), metal on bone is 0.5, metal on polyethylene is 0.1, metal on metal is 0.5, and metal on Teflon™ is 0.02. COF lowers with wettability, indicating a layer of fluid between surfaces decreases friction.

Suitable, but non-limiting, examples of low friction material include metal; metal alloy with an amorphous atomic structure (of which Liquidmetal® alloys from Liquidmetal® Technologies of Lake Forest, Calif. are representative); ceramics; ceramic-coated material; polymers, optionally reinforced with fiber; pyrolitic carbon coated material; carbon composites; and diamond-coated material. Preferred examples include stainless steel, cobalt-chrome alloy, titanium; titanium- and zirconium-based Liquidmetal® alloy; alumina, zirconium oxide; polyetheretherketones, polyetherketoneketones, polyaryletherketones, polysulfones; P25-CVD. Still more preferred examples include stainless steel, cobalt-chrome alloy, titanium, zirconium-based Liquidmetal® alloy, zirconium oxide, polyetheretherketones, polyetherketoneketones, polyaryletherketones, polysulfones, and P25-CVD.

Cobalt-chrome alloy has been used in joint replacement for over 30 years. It is the most common bearing surface in joint replacement surgery due to its strength, durability, biological tolerance, low reactivity, and relatively low friction articulation against polyethylene, the most common material against which it articulates. In spite of cobalt-chrome's long-term success, there are drawbacks to the use of this material. Cobalt-chrome articulating against polyethylene generates a low, but significant, amount of friction. In fact, it has been calculated by Bankston, et al. (The Comparison of Polyethylene Wear in Machined vs. Molded Polyethylene, CORR, 317:37-43, August 1995), that the linear wear rate for compression molded polyethylene is 0.05 mm/year and 0.11 mm/yr for ram extruded polyethylene, when cobalt-chrome is used with polyethylene.

Another class of low friction material used in joint replacement surgery is ceramics. The most common used are alumina and zirconia. Ceramics are advantageous over cobalt-chrome in that the wear rate against polyethylene is only 1-10% that of cobalt-chrome; the wear rate of ceramic on ceramic is even lower. Thus, ceramic surfaces have the potential for long term success with little wear. The problem with ceramics is their relative brittleness and potential for breakage. With advances in ceramic materials technology this problem has been nearly eliminated in hip replacement surgery, where the ceramic replacement of the femoral head and/or acetabular cup has shown little potential for breakage. However, due to the geometry of the knee joint and the difference in how forces are transmitted in the knee, ceramics have not found a role as joint replacement material for the knee joint.

A method is available in which a layer of zirconium oxide ceramic is formed on the surface of a zirconium metal alloy. The ceramic surface layer is desirable in that it exhibits lower friction and lower generation of heat at the articulating surface than metal alloy, yet the metal alloy maintains the strength, so that the relative brittleness of a zirconium ceramic is avoided. Several U.S. patents have been issued with regards to the zirconium oxide layer including U.S. Pat. Nos. 5,037,438, 5,180,394, and 6,447,550. Additionally, U.S. Pat. No. 6,206,927 discloses as an option that a steel-ceramic composite may be used instead of solid steel, (i.e. cobalt-chrome) for their UniSpacer™-type device.

An additional type of alloy that could be considered as the surface bearing material is currently being co-developed by DePuy and Liquidmetal® Technologies, Inc. Available data on their zirconium-based alloy suggests that it would have favorable properties for use as a surface bearing implant material. This includes hardness, low-friction, wear resistance, superior strength, and superior elastic limit. Representative patents for this type of material include U.S. Pat. Nos. 5,288,344 and 5,368,659 (to Caltech) and U.S. Pat. Nos. 5,567,251, 5,567,532, 5,866,254, and 6,818,078 to Liquidmetal® Technologies, Inc., all of which are incorporated by reference in their entirety.

The use of a diamond-coated surfaced has been demonstrated to exhibit a very low coefficient of friction; a diamond-like carbon (DLC) coating on cobalt-chrome metal has reduced wear of adjacent polyethylene. This is disclosed in U.S. Pat. No. 6,171,343, which claims the process of coating a metal alloy with DLC in order to further reduce friction. U.S. Pat. No. 6,800,095 is a representative patent for Diamicron, Inc. (Orem, Utah); Diamicron has several patents claiming a diamond surface in orthopedic implant devices. Lockheed Martin Corp. also has a diamond coating process that may be applied to biological implants. The use of a diamond coating is also described in U.S. Pat. No. 6,626,949 (to BioPro, Inc.).

Polyetheretherketone (PEEK) is a polymer that, with fiber reinforcement, results in a hard, durable, low-friction, low reactivity material. It has been mostly applied in spinal surgery where the material replaces titanium as an insert between vertebrae, giving stability and thus allowing for spinal fusion to occur. PEEK is one of several polymers, (others include polyetherketoneketone, PEKK, polyaryletherketone, PAEK, and polysulfones) that can be reinforced with fibers, such as carbon or glass, giving the polymers differing properties of strength, hardness, and flexibility. PEEK and related materials have been proposed for use in femoral implants and as intervertebral discs due to the capacity to achieve either a hard, low-friction surface or an elastomeric surface, depending on the fiber reinforcement pattern. The properties of low-friction, along with biocompatibility and strength, make PEEK and its related polymers potentially good candidates for use as material in the implant described herein. A hard outer composite can be mixed with a softer, more elastic, inner composite, which would confer the desired characteristics of the device herein, namely low-friction articulation and cushioning. The use of PEEK in orthopedic implants is represented by U.S. Pat. No. 6,673,075; furthermore, PEEK fibers have been developed by Zyex Corporation (Gloucester, UK).

Carbon-carbon composites have been suggested for use as material in orthopedic implants. This is due to their strength, biocompatibility, and low wear rates. One compound in particular, P25-CVD, exhibited a very low wear rate when tested for use as a total hip bearing.

Cobalt-chrome, ceramics and metal-ceramic composites all have a high modulus of elasticity (MOE) as compared to bone. This high MOE imparts inordinate stresses to the articulating bone. Zirconium alloy can be favorable over cobalt-chrome, for example, because its MOE is significantly lower. Cobalt-chrome's MOE is approximately 220 GPa, whereas zirconium alloy has a MOE on the order of 83-100 GPa; titanium has a MOE of approximately 110 GPa. All of these materials are far from subchondral bone, which has a MOE of approximately 2 GPa, whereas cortical bone has a MOE up to 17 GPa.

In order to find materials which better approximate the MOE of bone, implants made out of pyrolitic carbon have been described; however, they are limited to low-weight bearing joints such as the wrist. Pyrolitic carbon has a MOE between 10-35 GPa. While this overlaps that of cortical bone, it is still higher than that of subchondral bone. Nonetheless, a pyrolitic carbon implant could be advantageous due to its relatively low MOE. In fact, there are patents for pyrolitic carbon coated surfaces, such as U.S. Pat. No. 4,166,292, and for use of pyrolitic carbon as implant material, including U.S. Pat. Nos. 4,457,984, 5,534,033, 6,090,145, and 6,436,146.

In addition, pyrolitic carbon has a low coefficient of friction; one would expect low wear rates and low heat generation in the opposing articulating surface. This is supported by Kawalee, et al. (Evaluation of fibrocartilage regeneration and bone response at full-thickness cartilage defects in articulation with pyrolitic carbon or cobalt-chrome alloy hemiarthroplasties. J. Biomed. Res., 1998, 41(4): 534-540), who demonstrate that pyrolitic carbon is better tolerated compared to cobalt-chrome when used as a surface bearing material for articulation with cartilage tissue or damaged cartilage tissue. Surface cracks were seen in only 14% of the cartilage surfaces articulating against carbon, but 100% had cracks when articulating against cobalt-chrome. Furthermore, cartilage defects had an 86% regeneration rate when articulating against carbon, but only a 25% regeneration rate when articulating against cobalt-chrome.

Due to its favorable MOE and low coefficient of friction, pyrolitic carbon, or implants coated with this material, could be used for joint implants. Pyrolitic carbon is used in joint implants currently, but this use is limited to the hand and wrist joints. This limitation is due to the fact that pyrolitic carbon is simply not strong enough for the larger weight bearing joints. Pyrolitic carbon has the propensity for undergoing cyclic fatigue because cyclic crack growth is possible in this material. Thus, stress is a limiting factor in the use of this material in a weight bearing function because of the potential for breakage and failure of the implant.

However, due to the stress dissipation properties of the cushioning component, pyrolitic carbon may be used as the low friction component material of the knee implant; because the pyrolitic carbon does not act as the weight-bearing material in the device, the potential for breakage and failure are greatly reduced.

The final type of low friction bearing surface relates to a biological surface. By this is meant a surface which is coated with a substance that resembles the normal cartilage surface. It is well known that hyaluronic acid (HA) acts as the lubricant in articulating cartilage and that the outer surface of cartilage has an HA coating, intermixed with the PG/collagen matrix. The negatively charged surface molecules and HA lubricant act to repel each other, thereby decreasing contact between adjacent cartilaginous surfaces; this repulsion results in a low friction articulation.

The use of low friction coatings in medical applications is not new. Most commonly, these consist of an HA coating. They are most often used as coatings for catheters, catheter introducers and tubes. When these devices are HA coated they slide easily within blood vessels and other body orifices. Patents representative of such coatings are U.S. Pat. No. 6,160,032 and U.S. Pat. No. 6,387,450. In addition, there are several products on the market which utilize a process for HA coating for a wide variety of uses. One such product is called Lubril AST™, (U.S. Pat. No. 6,238,799). This product is meant to decrease the COF down to 0.009, which is nearly as good as the best cartilage-on-cartilage articulations. Although it demonstrates durability, this test is performed under “mild conditions;” this may not be the same as in actual joint articulation. Another such product is called HYDAK™, which is a registered trademark of Biocoat. This product claims to have, in addition to thickness, wettability, lubricity and low friction, abrasion resistance, and stability in contact with body fluids. Furthermore, this product may be applied to many different types of materials including polyurethane, PMMA, ceramics, titanium, and more.

(b) Cushioning Material

In practicing the invention, the phrase “cushioning” means the ability to absorb and dissipate weight bearing loads by deformation; cushioning in the context of the present invention means a material possessing a modulus of elasticity (MOE) between about 0.1 and 50 MPa. The cushioning material of the present invention is also preferably elastomeric. Elastomeric materials are those that deform when stressed with a load, but return to their original shape when the load is removed. Common elastomeric materials include rubber, synthetic rubber or polymer, and/or plastics. By way of example, the MOEs of some materials include: polyvinylalcohol (PVA) 0.5-10 MPa, rubber ˜7 MPa, and cartilage ˜24 MPa. Suitable, but non-limiting, examples of cushioning material include polyurethane, polyvinylalcohol, polyacrlyamide, fiber-reinforced polymer, and a water retaining center comprising a hydrogel made from a material selected from the group comprising polyvinylalcohol or polyacrylamide, surrounded by a tight outer covering. Preferred examples include polyurethane and a water retaining center comprising a hydrogel made from a material selected from the group comprising polyvinylalcohol or polyacrylamide, surrounded by a tight outer covering.

The cushioning material of the present invention is optionally made out of an elastomeric compound. The types of compounds that can be used include those made of a single material, such as polyvinyl alcohol, polyurethane and polyacrylamide; alternatively a device constructed from more than one material may be used. This could include a hydrogel material, which is surrounded by a tight, non-elastic covering.

U.S. Pat. No. 6,224,630 discloses a device for use in vertebral disc repair. PVA is the preferred material, but the patent discloses many materials including polyurethane, polyethylene, polypropylene, etc. U.S. Pat. No. 5,458,643 discloses an artificial intervertebral disc made out of a PVA hydrogel, with a ceramic or metal porous body; it also discloses PVA for use as an artificial articular cartilage repair material. U.S. Pat. Nos. 5,981,826 and 6,231,605 describe PVA for use as tissue scaffolding.

SaluMedica is marketing a product called SaluCartilage™, which is meant to be a cartilage defect replacement material. Salucartilage is made from a PVA polymer; it is described in U.S. Pat. No. 6,231,605, by David Ku, who is also the CEO and President of SaluMedica. This product's mechanical properties are similar to those of articular cartilage and it is capable of withstanding repetitive loading typical of normal walking conditions. It apparently has a very low friction when articulating against an opposing cartilage surface. Although the mechanical properties and strength appear to be adequate, this substance, when used as a bearing surface, has a relatively high coefficient of friction (COF). Covert and Ku demonstrate (in vitro) (Covert, R. J., and Ku, D. N., Friction and wear testing of a new biomaterial for use as an articular cartilage substitute. BED-Vol. 50, 2001 Bioengineering Conference, ASME 2001) that although the COF of their PVA material appears to be high, 0.184 against bovine cartilage and 0.247 against damaged articular cartilage (for comparison, cartilage on cartilage: 0.01-0.02; metal-on-metal: 0.15-0.35; metal on UHMWPE: 0.05-0.15), this level of friction does not have a direct relationship with wear and should not be used to predict wear rates. Even though it is stated that wear rates may not be a problem in spite of the high friction, one would have to be skeptical until in vivo testing determined that the high friction levels did not cause any problems on the adjacent normal cartilage. Importantly, the SaluCartilage™ device is only being tested as a cartilage defect replacement material, and not as a knee spacer.

Polyacrylamide has been used for many years in the human body. It has been used as an injectable filler for wrinkles and lip augmentation, and, in the past, as a breast implant filler; thus it has been deemed safe for human implantation (U.S. Pat. No. 5,941,909 to Mentor Corp.; filler for implants such as breast or testicles).

A disc implant from RayMedica is a hydrogel surrounded by a constraining jacket. (U.S. Pat. No. 5,824,093.) The implant material is made out of acrylamide and acrylnitrile. The second option disclosed in this patent is to use PVA as the hydrogel core, surrounded by a jacket made out of high molecular weight polyethylene weave. The mechanism of action is similar to that of articular cartilage: the core hydrogel material absorbs and releases fluid, similar to the PG component of articular cartilage ECM. The outer “jacket” limits excessive fluid absorption, not unlike the collagen type II effects in cartilage. This type of material, a core of hydrogel surrounded by an outer non-elastic material is proposed only for use in the spine as a disc replacement. There are no references to, nor any implications for, use elsewhere, as in the knee joint.

Polyurethane is well-known in industrial applications, i.e. wheels, etc., due to its favorable strength and wear properties. It is also known to be well-tolerated by the body, having been successfully employed as an implant for tendons, arteries, and veins.

In the early 1960s polyurethane was used to replace the acetabulum, but due to the poor quality of polyurethane available at that time, the implants essentially fell apart, and polyurethane for use in joint replacement was abandoned. In 2001 Townley was issued U.S. Pat. No. 6,302,916, for the use of polyurethane as a material in joint replacement, i.e. tibial tray and acetabular cup. Townley discloses that the polyurethane essentially performs the same function as does UHMWPE in conventional TKAs; it acts as the bearing surface between the fixed femoral and fixed tibial components. It is stated in that patent that the polyurethane has similar, if not better, wear properties than UHMWPE. An additional advantage is that polyurethane can be heat treated, whereas UHMWPE cannot, and thus it can be heat sterilized. It also has a longer shelf-life. The patent does not disclose the use of polyurethane in a UKA; the patent additionally does not describe, nor does it imply, the use of polyurethane in a manner where the tibial or femoral components are unattached to bone. Furthermore, no advantage with respect to smaller incisions or increase in activity, such as running, are described or implied. Thus, the polyurethane is merely a substitute for UHMWPE, with no further advantages such as smaller incision size, less surgical dissection, fewer bone cuts, or an increase in post-operative activity, as compared to a standard TKA using UHMWPE as the bearing surface against metal.

U.S. Pat. No. 6,248,131 to Felt, et al., discloses a polyurethane implant meant for intervertebral disc replacement. Because the polyurethane material articulates against degenerating cartilage with this device, it could be expected to demonstrate significant wear, and thus would not make an optimal implant due to the poor capacity as a low friction bearing material. Another patent issued to Felt, U.S. Pat. No. 6,652,587 discloses a knee implant, made out of an elastomeric material such as polyurethane, in which the tibial and femoral components are fixed to bone, unlike the present invention.

Impliant, Ltd. (Ramat Poleg, Israel) has developed a proprietary polycarbonate urethane compound for medical purposes. Specifically, they have developed a hip replacement implant, a femoral head replacement. This femoral prosthesis consists of a titanium stem for insertion into the femoral canal, similar to current femoral stems. A Morse taper is used on the neck component, onto which a titanium head can be attached, again, similar to other femoral head replacements. The implant is unique in that the titanium head is covered with an elastomeric component, which is meant to articulate against the adjacent acetabular cartilage. Prior femoral components do not have an elastomeric surface; rather the metal head articulates with the acetabular cartilage.

The Impliant elastomeric coating is a proprietary polycarbonate urethane material. Furthermore, the methods of manufacture and methods of attachment are also proprietary. This implant is meant for the hip only; the company literature gives no mention of a knee implant, even though it mentions other uses for polyurethanes in medical devices, including spinal disc implants, intra-aortic pumps, and pacemaker leads.

Impliant has described elastomeric implants in WO 2004/014261 (femoral head prosthesis), and WO 03/047470 (hip, shoulder, knee implants). With respect to the knee, the Impliant invention describes a meniscal replacement type of prosthesis; it is not used as an implant for arthritic joint replacement. Indeed, because the implant is C-shaped the center part allows for opposing joint surfaces to make contact, unlike the invention disclosed herein.

Of the above materials, polyurethane holds the most promise, stemming from its favorable rheological properties, tolerance by the body as an implant, low wear rate, and overall strength. A more physiological cushioning represented by an acrylamide hydrogel and with an inelastic outer covering is also a good option.

Manufacturing of the FLFC involves CAD/CAM (computer assisted design/computer assisted manufacturing) techniques. The overall shape of each femoral condyle for humans can be determined for numerous sizes, with a range of individuals from 90 lbs. to over 300 lbs. One millimeter to 1½ mm increments in the overall size of the implants can be used to provide all of the varying size ranges in humans. CAD/CAM techniques are used to create molds for these sizes. The implants can then be made within these molds and polished as needed. When the use of molds is not practical, CAD/CAM techniques can be used to machine the implants from a solid block. The machined implants are then polished as needed.

The CC is manufactured as described by prior art. U.S. Pat. No. 6,302,916, to Townley describes proprietary polyurethane, while U.S. Pat. Nos. 6,306,177 and 6,652,587 (to Advanced Bio-Surfaces, Inc.) describe a method of manufacturing a polyurethane implant. Impliant, Ltd. (Netanya, Isreal) is a company with a proprietary polyurethane material currently being used for a femoral head prosthesis. The Impliant material is described in numerous PCT patents, as represented by WO 03/047470. Alternative cushioning materials include PVA, which is described in U.S. Pat. No. 6,231,605, and PEEK, which involves the inclusion of a fiber mesh within the PEEK material in order to generate elastomeric properties.

The shape of the cushioning material is such that it matches each different size of the low friction implant. Mechanical interlocking is used to ‘lock’ and stabilize the cushioning material into the low friction portion of the implant.

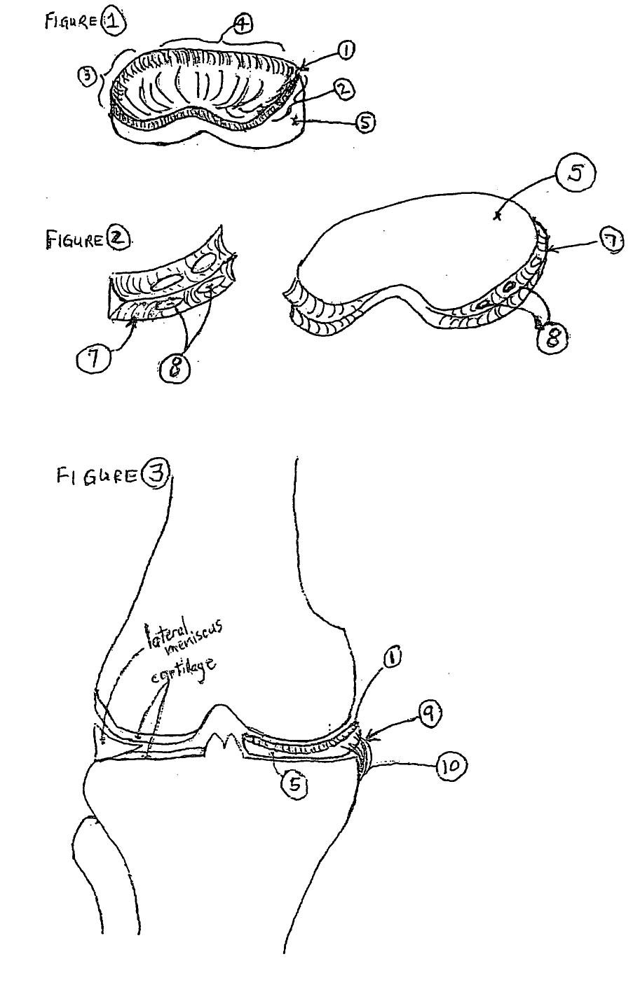

In one embodiment of the present invention, a prosthetic device is provided as a single structure, comprising two components: an upper low friction layer and a lower cushioning layer. It is intended that the prosthetic not be attached to the tibia. The upper layer is made out of a low friction material. Bound to the undersurface of the upper layer is the elastomeric cushioning component (CC). The upper, low friction layer is called the femoral low friction component (FLFC). It is contoured to match the shape of the femoral condyle. The CC, which is made out of an elastomeric material, is contoured on its superior surface to the exact dimensions of the undersurface of the FLFC in order that the two could be attached. The undersurface of the CC is generally flat with a slight convexity, in order to coincide with the relatively flat, slightly convex tibial articular surface. The contour is given a slight variation in order to better mimic the shape of the medial vs. the lateral tibial surface geometry. For example, FIG. 1 shows a perspective view of a representative two-piece construct. There is a top, or superior, piece (1), the FLFC (femoral low-friction component), that is made out of a low friction material. Its shape conforms to that of the femoral condyle. This shape resembles the general shape of the meniscus cartilage, but instead of forming a “C” shape with an open central/inner portion as in the normal meniscus, the central or inner portion is solid. The front (anterior) (2), back (posterior) (3), and side (lateral) (4), portions are raised to provide for some stability and also to add to the total surface area where weight load is transferred. The radius of curvature is equal to and/or preferably slightly greater than that of the opposing femoral condyle. Furthermore, the posterior portion is generally wider than is the anterior portion. The undersurface is attached to the elastomeric cushioning component (5). The CC (5) may be attached to the FLFC (1) by mechanical interdigitation, molecular fixation or glue. Mechanical interdigitation can include any one of a number of locking mechanisms, with or without the use of a separate ring or pin device that acts as the locking agent. Furthermore, the entire two-component construct may optionally be manufactured together, or the pieces may be manufactured separately where the surgeon attaches them together at the time of surgery. In this latter option a simple snap on mechanism may be used for attachment of the two components.

In an aspect of this embodiment, the FLFC is made from a material selected from the group comprising metal, metal alloy with an amorphous atomic structure (of which Liquidmetal® alloys from Liquidmetal® Technologies of Lake Forest, Calif. are representative), ceramic, glass, carbon composites, polymers, ceramic-coated surface materials, diamond-coated surface materials, pyrolitic carbon-coated surface materials.

In another aspect, the FLFC is made from metal. In a preferred aspect the metal is selected from the group comprising stainless steel, titanium, cobalt-chrome alloy.

In yet another aspect, the FLFC is made from ceramic. In a preferred aspect the ceramic is selected from the group comprising alumina, zirconium oxide.

In yet another aspect, the FLFC is made from carbon composite. In a preferred aspect the carbon composite is P25-CVD.

In yet another aspect, the FLFC is made from a polymer. In a preferred aspect the polymer is selected from the group comprising polyetheretherketone, polyetherketoneketone, polyaryletherketone, polysulfone.

In yet another aspect, the FLFC is made from a polymer optionally reinforced with fiber.

In yet another aspect, the FLFC is made from pyrolitic-carbon coated material.

In yet another aspect, the FLFC is made from a ceramic-coated material.

In yet another aspect, the FLFC is made from a diamond-coated material.

In yet another aspect, the FLFC is made from glass.

In yet another aspect, the FLFC is made from metal alloy with an amorphous atomic structure (of which Liquidmetal® alloys from Liquidmetal® Technologies of Lake Forest, Calif. are representative). In a preferred aspect the alloy is selected from the group comprising titanium-based Liquidmetal® alloy or zirconium-based Liquidmetal® alloy. In an even more preferred aspect the alloy is zirconium-based Liquidmetal® alloy.

In another aspect, the CC is made from an elastomeric material selected from the group comprising polyurethane, polyvinylalcohol, polyacrlyamide, fiber-reinforced polymer. In a preferred aspect the CC is made from polyurethane.

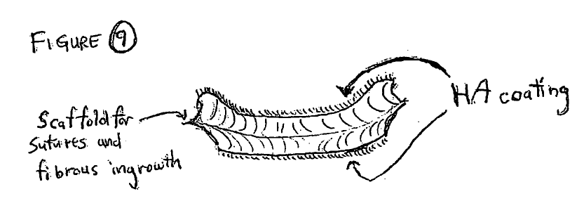

In yet another aspect, the CC is made from a capsule comprising a water retaining center surrounded by a supportive outer covering. In a preferred aspect the water retaining center is made from hydrogel material selected from the group comprising polyacrylamide and polyvinylalcohol. For example, FIG. 8A shows a representative hydrogel/tight outer coating option for the prosthesis. The superior surface has a FLFC as disclosed above. The undersurface has a TLFC, as disclosed above. The CC, instead of being composed of one elastomeric material, may consist of two parts: an inner hydrogel component and an outer water-permeable synthetic fiber component (14). The hydrogel has an affinity for water and will attract water inside, as noted by (15) in FIG. 8A. This constant inward flow of water puts outward pressure on the outer coating (14) and both the FLFC (1) and the TLFC (11), as depicted by the arrows inside the component. This constant inward flow of water is resisted by the outer coating (14). The inward force is constant because the outer coating is made smaller/tighter than the full expansile extent of the inner hydroge. This inward force is responsible for the cushioning effect. FIG. 8B demonstrates what would happen if the hydrogel (16) were not surrounded by the outer coating. Here the unimpeded inward flow of water causes the hydrogel to expand to a much larger size. The inward and outward water flow pressures equilibrate (17). FIG. 8C demonstrates what occurs with weight loads. The weight load (18) causes the thickness of the cushioning component to decrease (19). The outward flow of water increases beyond the inward flow (20). The inward flow of water, along with the tension created in the outer coating of fibers, resists complete outward flow of water. This resistance and the inward and outward flow of water are responsible for the cushioning properties. This mimics what occurs in normal hyaline cartilage, where cushioning is also provided by the inward and outward flow of water. In normal hyaline it is the PG portion of the matrix that acts as the hydrogel, attracting water into the matrix. The type II collagen fibers of the matrix resist tension, just as does the outer fibrous coating of the implant. The hydrogel may be composed of an acrylamide or PVA. The outer coating may be composed of non-elastic fibers, such as polyethylene. One skilled in the art will recognize that other materials will possess properties making them appropriate or desirable materials for use in the outer coating.

In yet another aspect, the prosthesis is suitable for attachment to surrounding soft tissue by the entire periphery of the implant. In a preferred aspect, the prosthesis is attached to the menisco-tibial ligaments. In yet another aspect, the prosthesis is suitable for attachment to surrounding soft tissue by only a portion of the periphery of the implant, including the anterior, medial/lateral, and/or posterior portion(s) of the implant. In a preferred aspect, the prosthesis is attached to the menisco-tibial ligaments. FIG. 2 is representative of the manner by which the periphery of the CC is to be attached to the menisco-tibial ligaments, with an area for initial suture attachment and later permanent fibrous ingrowth. The rim (7) of the CC (5) has a collagen ingrowth coating (7). Rings (8), or a suitable alternative, may be used for suture fixation, which gives initial stability before fibrous ingrowth takes place.

In yet another aspect, the prosthesis is suitable for initial attachment to surrounding soft tissue by glue or sutures.

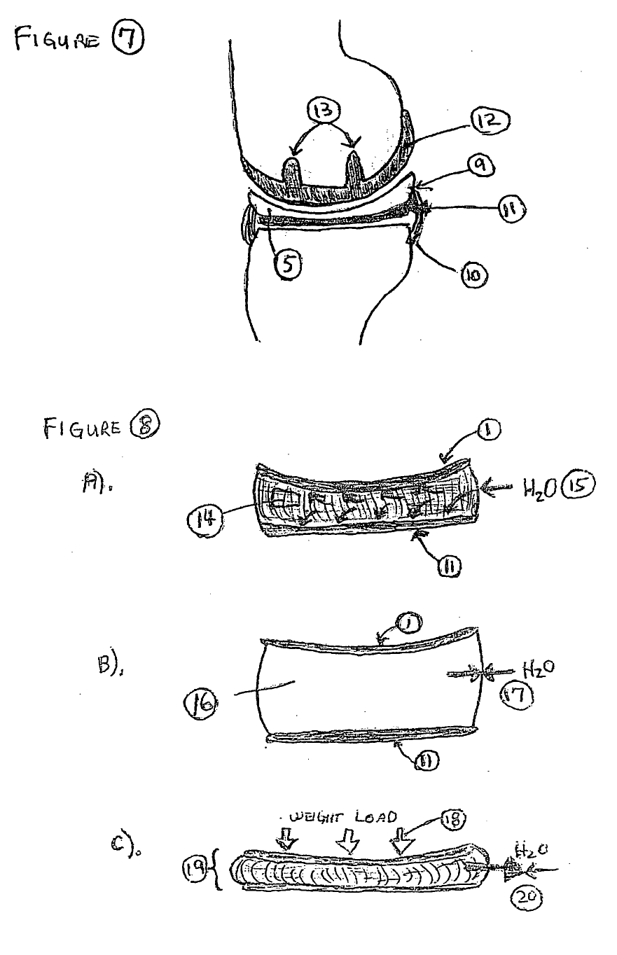

In yet another aspect, the CC further comprises a porous collagen ingrowth coating to facilitate permanent attachment via fibrous ingrowth. FIG. 6 shows the CC outer rim for initial biodegradable suture attachment and permanent fibrous ingrowth (9).

In yet another aspect, the FLFC is contoured to approximate the shape of the femoral condyle.

In yet another aspect, the FLFC has a radius of curvature equal to or larger than that of the femoral condyle against which it is intended to articulate. It is preferred that the FLFC has a radius of curvature greater than that of the femoral condyle against which it is intended to articulate.

In yet another aspect, the CC is contoured to exactly match the undersurface of the FLFC.

In yet another aspect, the CC is slightly larger than the FLFC. FIG. 6 shows an example of both of these aspects: the CC (5) may glide (see arrows pointing how the CC glides back and forth in the lateral view) on top of the tibial articular surface, guided by the attached menisco-tibial ligaments (10). The size of the CC is chosen so that it may articulate with the underlying tibial articular surface and with numerous different sizes of the attached FLFC.

In yet another aspect, the CC is attached to the FLFC by mechanical interdigitation, glue, or other bonding method.

In yet another aspect, the CC is attached to the FLFC prior to packaging.

In yet another aspect, the CC is attached to the FLFC immediately prior to implantation. In a preferred aspect the method of attachment of the CC to the FLFC is by a snapping mechanism.

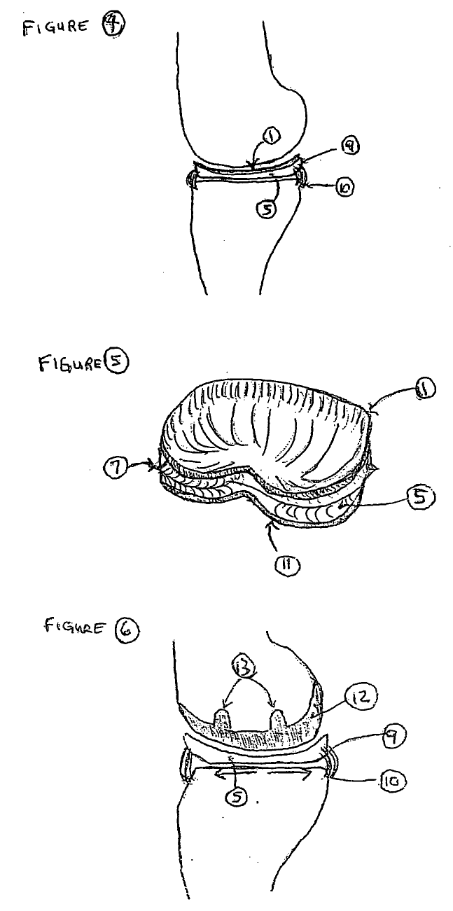

In yet another aspect, the prosthesis comprising a single structure, of three components: an upper low friction layer, a middle cushioning layer and a lower low-friction layer; wherein it is intended that the prosthetic not be attached to the tibia or the femur; the upper layer is made out of a low friction material; bound to the undersurface of the upper layer is the elastomeric cushioning component (CC); the upper, low friction layer is called the femoral low friction component (FLFC); it is contoured to match the shape of the femoral condyle; the CC, which is made out of an elastomeric material, is contoured on its superior surface to the exact dimensions of the undersurface of the FLFC in order that the two could be attached; the undersurface of the CC is generally flat with a slight convexity, in order to coincide with the relatively flat, slightly convex tibial articular surface; the contour is given a slight variation in order to better mimic the shape of the medial vs. the lateral tibial surface geometry; further comprises a tibial low friction component (TLFC), said component being attached to the undersurface of the cushioning component. For example, the CC may optionally have a low friction material attached to its undersurface. In this way the tibial articular surface articulates against a low friction bearing surface, rather than against the CC material, where there is the potential for wear of the CC component. FIG. 5 demonstrates a perspective view of the representative single unit as a three-piece combined construct. Here there is a top, superior, piece (1), the FLFC. The components may be manufactured as one single unit, or they may be separate pieces that are put together by the surgeon at the time of surgery. The CC has an outer rim for initial biodegradable suture attachment (7) and for later permanent fibrous ingrowth (7). The tibial low friction component, TLFC (11) may be attached to the undersurface of the CC. Its superior surface is the same size and shape as the undersurface of the CC. If attached, it is attached to the CC just as the FLFC is attached. The undersurface, or lower surface, of the TLFC is relatively flat to coincide with the tibial articular surface. Alternately, the under surface may be gently curved as is the tibial surface. This implant is inserted between the two articular surfaces just as in FIG. 3.

In yet another aspect, the TLFC is attached to the cushioning component-femoral low friction component unit by mechanical interdigitation, glue, or other bonding method.

In yet another aspect, the TLFC is attached to the cushioning component-femoral low friction component unit prior to packaging.

In yet another aspect, the TLFC is attached to the cushioning component-femoral low friction component unit immediately prior to implantation. In a preferred aspect the method of attachment of the TLFC to the CC is by a snapping mechanism.

In yet another aspect, the prosthesis components are optionally coated with hyaluronic acid. The hyaluronic acid coating may be applied to the hard, low friction components (FLFC and/or TLFC), to the cushioning elastomeric component, or both types of components; this is depicted in FIG. 9.

In yet another aspect, the FLFC is suitable for attachment to the femoral condyle. In a preferred aspect the FLFC is suitable for attachment to the femoral condyle by bone cement or by use of a porous coating, and/or hydroxy-apatite coating on the implant which allows for bone ingrowth into the implant. FIG. 6 demonstrates a lateral view of representative attachment of the FLFC (12) to the femoral condyle. It may be attached by either the use of bone cement or by bone ingrowth into a porous coated attachment surface on the FLFC (12). Pegs (13) are added in order to increase fixation stability of the implant into the femoral bone. The bone is cut according to a guiding jig. The proper sized component is inserted into place where it fits with contact on all attachment surfaces.

In yet another aspect, the FLFC is coated with an elastomeric or cushioning material (e.g. polyurethane).

In another embodiment of the present invention, a prosthetic device is provided as two components which are not attached to each other: an upper low friction layer and a lower cushioning layer. It is intended that the prosthesis not be attached to the tibia, but one component is attached to the femur. The upper layer is made out of a low friction material; its superior surface is made to attach to the femoral condyle. The upper, low friction layer is called the femoral low friction component (FLFC). Below the upper layer is the elastomeric cushioning component (CC); its upper surface is contoured to match the shape of the overlying FLFC, against which it articulates. The undersurface of the CC is generally flat with a slight convexity, in order to coincide with the relatively flat, slightly convex tibial articular surface. The contour is given a slight variation in order to better mimic the shape of the medial vs. the lateral tibial surface geometry.

In an aspect of this embodiment, the FLFC is made from a material selected from the group comprising metal, metal alloy with an amorphous atomic structure (of which Liquidmetal® alloys from Liquidmetal® Technologies of Lake Forest, Calif. are representative), ceramic, glass, carbon composites, polymers, ceramic-coated surface materials, diamond-coated surface materials, or pyrolitic carbon-coated surface materials.

In yet another aspect, the FLFC is made from metal. In a preferred aspect the metal is selected from the group comprising stainless steel, titanium, or cobalt-chrome alloy.

In yet another aspect, the FLFC is made from ceramic. In a preferred aspect the ceramic is selected from the group comprising alumina, or zirconium oxide.

In yet another aspect, the FLFC is made from carbon composite. In a preferred aspect the carbon composite is P25-CVD.

In yet another aspect, the FLFC is made from a polymer. In a preferred aspect the polymer is selected from the group comprising polyetheretherketone, polyetherketoneketone, polyaryletherketone, or polysulfone.

In yet another aspect, the FLFC is made from a polymer optionally reinforced with fiber.

In yet another aspect, the FLFC is made from pyrolitic-carbon coated material.

In yet another aspect, the FLFC is made from a ceramic-coated material.

In yet another aspect, the FLFC is made from a diamond-coated material.

In yet another aspect, the FLFC is made from glass.

In yet another aspect, the FLFC is made from metal alloy with an amorphous atomic structure (of which Liquidmetal® alloys from Liquidmetal® Technologies of Lake Forest, Calif. are representative). In a preferred aspect, the alloy is selected from the group comprising titanium-based Liquidmetal® alloy or zirconium-based Liquidmetal® alloy. In an even more preferred aspect the alloy is zirconium-based Liquidmetal® alloy.

In yet another aspect, the CC is made from an elastomeric material selected from the group comprising polyurethane, polyvinylalcohol, polyacrlyamide, or fiber-reinforced polymer. In a preferred aspect the CC is made from polyurethane.

In yet another aspect, the CC is made from a capsule comprising a water retaining center surrounded by a supportive outer covering. In a preferred aspect, the water retaining center is made from hydrogel material selected from the group comprising polyacrylamide and polyvinylalcohol.

In yet another aspect, the prosthesis is suitable for attachment to surrounding soft tissue by the entire periphery of the implant. In a preferred aspect, the prosthesis is attached to the menisco-tibial ligaments.

In yet another aspect, the prosthesis is suitable for attachment to surrounding soft tissue by only a portion of the periphery of the implant, including the anterior, medial/lateral, and/or posterior portion(s) of the implant. In a preferred aspect, the prosthesis is attached to the menisco-tibial ligaments.

In yet another aspect, the prosthesis is suitable for initial attachment to surrounding soft tissue by glue or sutures.

In yet another aspect, the CC further comprises a porous collagen ingrowth coating that facilitates permanent attachment via fibrous ingrowth.

In yet another aspect, the femoral condyle is cut to exactly match the superior surface of the FLFC, which is suitable for binding with bone cement.

In yet another aspect, the femoral condyle is cut to exactly match the superior surface of the FLFC, which is porous coated or hydroxy-apatite coated to allow for bone ingrowth.

In yet another aspect, the undersurface of the FLFC is polished in order to generate a low friction surface.

In yet another aspect, the CC is contoured to exactly match the undersurface of the FLFC.

In yet another aspect, the CC is slightly larger than the FLFC.

In yet another aspect, the prosthesis comprising two components, which are not attached to each other: a separate upper low friction component, and a single lower component consisting of two materials, a superior cushioning layer which is attached to a lower low-friction layer; wherein it is intended that the prosthetic not be attached to the tibia, but one component is attached to the femur; the upper low friction component is made out of a low friction material. Its superior surface is made to attach to the femoral condyle. The upper, low friction component is called the femoral low friction component (FLFC). Below the upper FLFC layer is the superior part of the lower component, consisting of an elastomeric cushioning component (CC). Its upper surface is contoured to match the shape of the overlying FLFC, against which it articulates. The undersurface of the CC is generally flat with a slight convexity, in order to coincide with the relatively flat, slightly convex tibial articular surface. The contour is given a slight variation in order to better mimic the shape of the medial vs. the lateral tibial surface geometry; further comprises a tibial low friction component (TLFC), said superior surface of said component being attached to the undersurface of the cushioning component.

In yet another aspect, the TLFC is attached to the cushioning component by mechanical interdigitation, glue, or other bonding method.

In yet another aspect, the TLFC is attached to the cushioning component prior to packaging.

In yet another aspect, the TLFC is attached to the cushioning component immediately prior to implantation. In a preferred aspect, the method of attachment of the TLFC to the CC is by a snapping mechanism.

In yet another aspect, the prosthesis components are optionally coated with hyaluronic acid.

In yet another aspect, the FLFC is suitable for attachment to the femoral condyle. In a preferred aspect, the FLFC is suitable for attachment to the femoral condyle by bone cement or by use of a porous coating, and/or hydroxy-apatite coating on the implant which allows for bone ingrowth into the implant.

In yet another aspect, the FLFC is coated with an elastomeric or cushioning material (e.g. polyurethane).

In yet another embodiment, there is provided a method of providing a knee prosthesis to a patient in need thereof, said method comprising: ascertaining the size and shape of the required prosthesis and components thereof by examination of the patient; and providing to the patient a prosthesis according to the present invention.

In yet another embodiment, there is provided a method of knee reconstruction of a patient in need thereof, said method comprising: determining the proper size and shape of a prosthesis and components thereof according to the present invention, by examination of the patient; selecting the prosthesis according to the present invention of said proper size and shape; exposing the knee compartment; and implanting the knee prosthesis into the compartment. The tibial articular surface may at times have irregularities. The tibial spines, which are located toward the center of the joint, may at times encroach upon the medial or lateral compartment. It is within the scope of this invention that the tibial articular surface may have to be shaved, or straightened out, in order to obtain proper and optimal prosthetic gliding without impingement upon the spines.

In yet another embodiment there is provided a method of making a prosthesis of the present invention comprising CAD/CAM design of molds for casting the prosthesis component.

In yet another embodiment there is provided a method of making a prosthesis of the present invention comprising CAD/CAM techniques to directly machine the components from blocks of material.

In yet another embodiment there is provided a kit for treating arthritis of the knee comprising a prosthesis of the present invention and means for implanting said prosthesis.

In yet another embodiment there is provided a method of implanting the prosthesis of the present invention, wherein the prosthesis is inserted between the femoral and tibial surfaces. FIG. 3 demonstrates a frontal view of a representative manner by which the implant may be inserted between the femoral and tibial articular surfaces. Fibrous ingrowth from the peripheral menisco-tibial ligaments (10) is demonstrated (9). FIG. 4 is a lateral view of a representative manner by which the implant is inserted between the femoral and tibial articular surfaces.

In yet another embodiment, numerous sizes of the components are provided so as to provide a prosthetic device appropriate for a given patient.

1. A knee prosthesis comprising:
(a) an upper, femoral low friction component; and
(b) a lower, cushioning component;
wherein said femoral low friction component faces a surface of a femur and said lower cushioning component faces a surface of a tibia, and wherein said prosthesis is not attached to the tibia.

2. The knee prosthesis of claim 1 wherein the upper femoral low friction component and lower cushioning component are associated in a single structure.

3. The knee prosthesis of claim 1 wherein the upper femoral low friction component and lower cushioning component are not associated in a single structure.

4. The knee prosthesis of claims 1, 2, or 3, further comprising a tibial low friction component, wherein said tibial low friction component is attached to the undersurface of the cushioning component.

5. The prosthesis of claim 1 wherein the femoral low friction component is made from a material selected from the group consisting of metal, metal alloy, ceramic, glass, carbon composites, polymers, ceramic-coated surface materials, diamond-coated surface materials, and pyrolitic carbon-coated surface materials.

6. The prosthesis of claim 5 wherein the metal is selected from the group consisting of stainless steel, titanium, and cobalt-chrome alloy.

7. The prosthesis of claim 5 wherein the ceramic is selected from the group consisting of alumina and zirconium oxide.

8. The prosthesis of claim 5 wherein the carbon composite is P25-CVD.

9. The prosthesis of claim 5 wherein the polymer is selected from the group consisting of polyetheretherketone, polyetherketoneketone, polyaryletherketone, and polysulfone.

10. The prosthesis of claim 9 wherein the polymer is fiber-reinforced.

11. The prosthesis of claim 4 wherein the femoral and tibial low friction components are made of material having a coefficient of friction of from about 0.001 to about 0.5.

12. The prosthesis of claim 11 wherein the femoral and tibial low friction components are made of material having a coefficient of friction of from about 0.001 to about 0.2.

13. The prosthesis of claim 11 wherein the femoral and tibial low friction components are made of material having a coefficient of friction of from about 0.001 to about 0.1.

14. The prosthesis of claim 5 wherein the metal alloy has an amorphous atomic structure.

15. The prosthesis of claim 14 wherein the metal alloy is titanium-based or zirconium-based.

16. The prosthesis of claim 1 wherein the cushioning component is made from an elastomeric material.

17. The prosthesis of claim 16 wherein the material is selected from the group consisting of polyurethane, polyvinylalcohol, polyacrlyamide, and fiber-reinforced polymer.

18. The prosthesis of claim 17 wherein the material is a polyurethane.

19. The prosthesis of claim 1 wherein the cushioning component is made from a capsule comprising a water retaining center surrounded by a supportive outer covering.

20. The prosthesis of claim 19 wherein the water retaining center is made from hydrogel material.

21. The prosthesis of claim 20 wherein the hydrogel material is polyacrylamide or polyvinylalcohol.

22. The prosthesis of claim 1 wherein the prosthesis is suitable for attachment to surrounding soft tissue along at least a portion of its periphery.

23. The prosthesis of claim 22 wherein the prosthesis is suitable for attachment to the menisco-tibial ligaments.

24. The prosthesis of claim 22 wherein the prosthesis is suitable for attachment to surrounding soft tissue by glue or sutures.

25. The prosthesis of claim 1 wherein the cushioning component further comprises a porous collagen ingrowth coating.

26. The prosthesis of claim 25 wherein the prosthesis is suitable for attachment to surrounding soft tissue by fibrous ingrowth.

27. The prosthesis of claim 2 wherein the femoral low friction component is contoured to approximate the shape of the femoral condyle.

28. The prosthesis of claim 27 wherein the femoral low friction component has a radius of curvature equal to or larger than that of the femoral condyle against which it is intended to articulate.

29. The prosthesis of claim 2 wherein the superior surface of the cushioning component is contoured to match the undersurface of the femoral low friction component.

30. The prosthesis of claim 2 wherein the cushioning component is attached to the femoral low friction component by mechanical interdigitation, glue, or other bonding method.

31. The prosthesis of claim 30 wherein the cushioning component is attached to the femoral low friction component prior to packaging.

32. The prosthesis of claim 30 wherein the cushioning component is attached to the femoral low friction component immediately prior to implantation.

33. The prosthesis of claim 30 wherein the attachment is achieved by a system which fastens the two components together.

34. The prosthesis of claim 3 wherein the femoral condyle is cut such that the superior surface of the femoral low friction component makes contact with the cut surface of the bone.

35. The prosthesis of claim 34 wherein the femoral low friction component is suitable for attachment to the femoral condyle.

36. The prosthesis of claim 35 wherein the femoral low friction component is suitable for attachment to the femoral condyle by a coating on the implant which allows for bone ingrowth into the implant.

37. The prosthesis of claim 36 wherein the coating comprises bone cement, hydroxy apatite coating, or a porous coating.

38. The prosthesis of claim 36 wherein the superior surface of the cushioning component has a radius of curvature equal to or larger than that of the femoral low friction component against which it is intended to articulate.

39. The prosthesis of claim 4 wherein the tibial low friction component is attached to the cushioning component-femoral low friction component unit by mechanical interdigitation, glue, or other bonding method.

40. The prosthesis of claim 4 wherein the tibial low friction component is attached to the cushioning component-femoral low friction component unit prior to packaging.

41. The prosthesis of claim 4 wherein the tibial low friction component is attached to the cushioning component-femoral low friction component unit immediately prior to implantation.

42. The prosthesis of claim 41 wherein the attachment is achieved by a system which fastens the tibial low friction component to the cushioning component-femoral low friction component unit.

43. The prosthesis of claims 1, 2, or 3 wherein the undersurface of the cushioning component is either flat or slightly concave, so as to match the convexity of the tibial surface against which it articulates.

44. The prosthesis of claim 4 wherein the undersurface of the tibial low friction component is either flat or slightly concave, so as to match the convexity of the tibial surface against which it articulates.

45. The prosthesis of claim 1 further comprising a coating of hyaluronic acid.

46. The prosthesis of claim 4 further comprising a coating of hyaluronic acid on any or all of the components.

47. A method of providing a knee prosthesis to a patient in need thereof, said method comprising:
(a) ascertaining the size and shape of the required prosthesis and components thereof by examination of the patient; and
(b) providing to the patient a prosthesis according to claim 1.

48. A method of knee reconstruction of a patient in need thereof, said method comprising:
(a) determining the proper size and shape of a prosthesis and components thereof according to claim 1, by examination of the patient;
(b) selecting the prosthesis according to claim 1 of said proper size and shape;
(c) exposing a knee compartment of the patient; and
(d) implanting the knee prosthesis into the compartment.

49. The method according to claim 48, wherein the prosthesis is implanted between the femoral and tibial surfaces.

50. A method of treating arthritis of the knee joint comprising replacement of damaged meniscal tissue with the prosthesis of claim 1.

51. A kit for treating arthritis of the knee comprising the prosthesis of claim 1 and means for implanting said prosthesis.
